(19) United States
(12) Patent Application Publication
(10) Pub. No.: US 2012/0277521 A1
(43) Pub. Date: Nov. 1, 2012

(54) Systems And Methods For Eliciting A Therapeutic Zone

(76) Inventor: D. E. Chamberlin, Glastonbury, CT (US)

(21) Appl. No.: 13/460,148

(22) Filed: Apr. 30, 2012

Related U.S. Application Data
(60) Provisional application No. 61/481,133, filed on Apr. 29, 2011.

(51) Int. Cl. A61M 21/02

(52) U.S. Cl. 600/28; 600/26; 600/27

(57) ABSTRACT

The systems, devices, and methods of the invention provide a solution to the problem of inefficiencies associated with the psychotherapeutic process. Heart rate variability (HRV) and other physiologic parameters can be used to regulate physiological state in the “real time” of the psychotherapy hour.

### RELATED APPLICATIONS

This application claims the benefit of the filing date of U.S. Provisional Application No. 61/481,133, which was filed on Apr. 29, 2011, the contents of which are incorporated herein by reference.

### FIELD OF INVENTION

The present invention relates generally to systems and methods for physiological psychotherapy and more particularly to systems and methods for eliciting a therapeutic zone during a physiological psychotherapy session.

### BACKGROUND

Although psychotherapy often demonstrates a significant degree of effectiveness in helping individuals overcome their presenting symptoms, the efficiency of the treatment may not measure up to the effectiveness. Psychotherapy can be very helpful, however progress is often uneven. A common factor is clients not being in the “right mood” or “frame of mind” to be able to engage and benefit from treatment. For example a person may be so anxious and preoccupied that she can't focus effectively. Or in talking about emotionally charged issues she may become overwhelmed, frozen and shut down.

### SUMMARY OF INVENTION

The systems, devices, and methods of the invention provide a solution to the problem of inefficiencies associated with the psychotherapeutic process. Heart rate variability (HRV) and other physiologic parameters can be used to regulate physiological state in the “real time” of the psychotherapy hour in a variety of ways. Clients can be instructed to use HRV immediately before a session while sitting in the parking lot, or in the waiting room. By beginning a session with an optimal level of arousal and focus, it is possible to “hit the ground running” making for a much more efficient and productive session.

In one aspect, the invention provides a system, the system including at least one sensor for detecting physiological information of a target, an input module coupled to the at least one sensor for receiving and processing the physiological information of the target, a central module running on a host computer coupled to the input module for further processing the physiological information of the target, and an output module coupled to the central module on the host computer for regulating physiological state of the target.

In one aspect, data is received characterizing a heart rate variability of a target. Determining, from the received data, a psychological state of the target and providing feedback to the target to elicit entry into a therapeutic zone.

In one aspect, a heart rate variability of a target is monitored. Breathing pattern instructions for the target is iteratively adjusted based on the heart rate variability thereby eliciting a resonance frequency to shift the psychological state of the target into a therapeutic zone. The resonance frequency being a large heart rate oscillation.

Implementations of the invention may provide one or more of the following features. The input module in the system encodes at least some of the physiological information of the target received from the at least one sensor. The central module in the system displays and stores the processed physiological information of the target. The output module in the system is configured to elicit a therapeutic zone from the target. The therapeutic zone can occur during a psychotherapy session.

Implementations of the invention may also provide one or more of the following features. The physiology information of the target includes one or more of the following: electromyographic information, electroencephalographic information, electrocardiographic information, respiration waveform, respiration rate, respiration amplitude, blood volume pulse waveform, heart rate, heart rate variability, skin temperature, and skin conductance. The output module in the system can generate an audio signal, a visual signal, and/or a mechanical signal.

The system is used to catalyze psychotherapy leading to an increase in the efficiency and/or effectiveness of the treatment. Accordingly, a method for optimizing a psychotherapy session or shifting a state of social engagement is carried out by detecting a breathing pattern of a subject and administering to the subject a signal during the therapy session to elicit resonance frequency of the cardiovascular system. A change in the breathing pattern to resonance frequency optimizes the psychotherapy session or shifts the state of social engagement into a pro-social mode. Alternatively, the steps of the method are carried out before or after the actual therapy session, i.e., the therapy hour.

The signal comprises an auditory, tactile, or visual stimulus. For example, a visual stimulus takes the form of an image on a wide screen television. The patient views an image such as a breathing pace in the shape of a moving ball or other object. The therapist manipulates the rate of the pacer to achieve the desired result (physiological and psychological response) from the patient.

The method utilizes a physiological response monitor and a program, e.g., software, that administers bilateral stimulation. Optionally, the method includes eye movement desensitization and reprocessing (EMDR). The method leads to a change in breathing pattern to a rate of 4-7 breaths per minute. The method involves the therapist engaging the subject to be treated at both a psychological level and at a physiological level. Stimulation and detection or monitoring of a physiologic response are done sequentially.

Also within the invention is a kit, which comprises the system/device assembly described above and instructions for using heart rate variability to train an individual to use breathing to stimulate the cardiovascular system at its unique resonance frequency for a sustained period of time.

The system and method leads to clinical benefit of the subject. For example, the method leads to increased well being, calmness, and health of the individual. Moreover, the therapy session is rendered more efficient in that the time to access and talk about a traumatic event (or otherwise disturbing or distressful state or event) is reduced.

Other features and advantages of the invention will be apparent from the following description of the preferred embodiments thereof, and from the claims. All references cited herein are incorporated by reference.

### BRIEF DESCRIPTION OF THE DRAWINGS

FIG. 1 is a block diagram of an exemplary system according to one embodiment of the invention.

FIG. 2 is a schematic view of an exemplary arrangement for eliciting a therapeutic zone.

FIG. 3 is a flowchart illustrating an exemplary process of eliciting a therapeutic zone.

FIG. 4 is an example display for providing instructions to a target relating to breathing rate.

FIG. 5 is a plot showing the time domain series of the magnitude of the heart rate variability and its rhythm.

FIG. 6 is a plot illustrating a time domain series of the magnitude of the heart rate variability and rhythm of a target after the target has been breathing at resonant frequency for several minutes.

### DETAILED DESCRIPTION

HRV has much to offer the individual who is trying to optimize function. From enhanced focus, to greater resilience and balance, HRV holds great promise. However the potential of HRV is often not realized in clinical practice because of certain unique challenges that this powerful technology presents to clinicians and clients. In particular the emergence of “unfinished business” from difficult past experiences can create an aversion to HRV feedback itself. As a result both parties lose enthusiasm, and a valuable opportunity for significant personal growth is lost.

HRV is used to train an individual to use breathing to stimulate her cardiovascular system at its unique resonance frequency for a sustained period of time. The resonance frequency is that rate of breathing, usually between 4 and 7 breaths per minute, which produces the largest heart rate oscillations, that is the greatest heart rate variability. This property makes it significantly more powerful than standard “Deep Breathing” techniques. Breathing at the resonance frequency stimulates pressure sensitive vascular baro-receptors, whose output ascends via the vagus nerve to modulate CNS function.

### Heart Rate Variability as an Adjunct to Psychotherapy

Physiological effects of HRV particularly relevant to psychotherapy include balancing of the autonomic nervous system, with an increase in parasympathetic tone. In addition there is increased production of synchronous alpha waves on the EEG. Psychologically this translates into a client who is “relaxed and ready” with an optimal level of arousal and focus. Internal dialog is reduced, feelings of well-being increased, and receptivity to input enhanced, each of which represents a clear benefit for psychotherapy.

### Resonance Frequency Training

Resonant Frequence Training is a variation of HRV biofeedback. Every individual has a “resonance frequency” at which heart rate variability is the greatest, and this resonance frequency can be measured with biofeedback instruments. While there is no uniform or ideal for all individual, this resonance frequency is often produced by a subjects in a relaxed mental state, with positive emotional tone, breathing diaphragmically and smoothly at a rate of about 4 to 7 breaths per minute. For example, relaxed breathing at about 6 b/m produces a spike of heart rate variability at about 0.1 Hz and tends to maximize most other measures of heart rate variability in most people. Identification of the specific breathing rate that will absolutely maximize heart rate variability measures for each individual patient (i.e., their individual Resonance Frequency) and training them to breathe diaphragmically at their Resonance Frequency optimizes clinical effects. Thus, psychophysiological balance improved (and a pro-social state achieved) by breathing at their Resonant Frequency. Exemplary resonance frequency comprises a breathing rate of 4, 4.5, 5, 5.5, 5.7, 6.0, 6.2, 6.5, 7.0.

### HRV Biofeedback Induced Adaptive Integration of Experience

Trauma is physically and/or psychologically threatening. When perceived threat crosses a threshold, the autonomic nervous system (ANS) is activated to provide the appropriate somatovisceral physiological support for one of three defensive modes—freeze, fight or flight.

The freeze response is mediated by the “vegetative vagus” nerve, and is characterized by immobilization, bradycardia, shift to external orientation and dissociation. [The vegetative vagus is the older of two parasympathetic systems in mammals, emanating from the dorsal motor nucleus. Porges-Polyvagal Theory. (Porges, S. W. (1995). “Orienting in a defensive world: mammalian modifications of our evolutionary heritage. A Polyvagal Theory.”Psychophysiology 32(4): 301-318; Porges, S. W. (2003). “Social engagement and attachment: a phylogenetic perspective.” Ann N Y Acad Sci 1008: 31-47.)]. Phylogenetically more recent is the sympathetic fight or flight response characterized by increased arousal, tachycardia, narrow focus attention etc.

When a supra-threshold threat has triggered a defensive response, fear conditioning via thalamo-amygdalar pathways ensure that similar stimuli presented in the future will non-consciously activate the ANS for rapid defensive response to facilitate survival. This response is most adaptive when it can be integrated with higher order e.g. cognitive function via thalamo-cortical pathways to allow for greater discrimination of triggering stimuli, appreciation of context, modulation of response etc. The adaptive integration of experience requires several critical conditions, including perception of safety.

The stress response turns off nonessential functions e.g. digestion/reproduction/repair to maximize survival under threatening conditions. Adaptive integration of experience is a psycho-physiological form of comprehensive systemic repair. When conditions are no longer threatening, these functions, including adaptive integration may resume. However if the individual does not have the psycho-physiological experience of safety, either because the environment remains hostile, or the ANS remains in one of the three defensive modes, adaptive integration will not take place.

The most recent evolutionary development in the regulation of the ANS is the ventral vagal complex, part of the “Social Engagement System”. The core of this system is the cortico-bulbar nuclei in the medulla which serve to facilitate social behavior by controlling ocular gaze, vocalization and the muscles of facial expression. Through the output of the myelinated “Smart” vagus nerve, this system puts the viscera in a prosocial state supporting “peaceful proximity”. If someone makes eye contact, smiles, and speaks to us softly we have a “gut feeling” of safety. This psycho-physiological state is reflected by robust heart rate variability.

The CNS is a nonlinear dynamic system which exhibits asymmetric reciprocal causality. This means that many elements are bidirectional including the “special visceral efferents” of the social engagement system. Increases in heart rate variability are caused by, and cause, activation of the social engagement system (personal communication).

As is described herein, heart rate variability biofeedback facilitates conscious control of the ANS and is useful as a somatic/ “bottom up” intervention which directly places the ANS in a psycho-physiological state conducive to the adaptive integration of experience. This state significantly reduces the propensity for dissociation which often compromises exposure therapies. When paired with Eye Movement Desensitization and Reprocessing (EMDR), individuals with un-integrated trauma experience rapid psycho-physiological functional reorganization.

This psycho-physiological state of “coherence” is characterized by a number of features:

1. Balanced oscillatory activation of the sympathetic/parasympathetic systems (which is distinct from other “relaxation techniques” e.g. Relaxation response, which cause a tonic increase in parasympathetic tone relative to sympathetic tone, but decrease overall autonomic output.)

2. “Centering” of the EEG to the alpha state which supports integration of diverse cortical and subcortical regions. This avoids the focal desynchronized processing of beta frequencies, and the dissociation characteristic of theta frequencies.

3. Myelinated vagus mediated ascending visceral regulation places the thalamus in “burst mode” (vs. transmission mode) acting as a gate to reduce excessive thalamo-cortical looping.

4. Psycho-physiological state of safety with activation of the social engagement system increases permeability of interpersonal boundaries with spontaneous increase in expression. In addition, the individual's ANS is more accessible for regulation by others. (After HRV, one patient reported—“Do I feel good because you're so calm?” A new experience for her.)

5. Places the system in a metastable state which is a resilient, adaptive state. HRV widely held to be an index of physiological adaptability.

6. Dissipation of pathologically stored excess energy in the system. (HRV biofeedback induces a state of systemic resonance by entraining multiple physiological oscillators including heart, respiration, baroreceptors, enteric pacemaker, thalamus etc. Putting a system at its resonant frequency creates a portal for energy transfer e.g. hit a metal pipe on a rock and the kinetic energy leaves as a tone at the resonant frequency.)

7. Turns off stress response (HPA axis). 23% reduction in cortisol after four weeks.

8. Breathing at the resonance frequency of the cardiovascular system (HRV) leads to a state of moderate arousal, with balanced activation of the autonomic nervous system, and increased alpha waves. These are the essential attributes of the “arousal portal” described by Les Fehmi, in which attention is fluid, and multiple attentional states co-exist (Fehmi, 2010).

### Clinical Observations of HRV Relevant to Psychotherapy/EMDR

1. Rapid shifts (15 min.) in physiological/psychological state. In non-trauma patients this is consistently experienced as a positive shift.

2. Induction of an apparent mixed state of relaxation and increased energy/anxiety/somatic sensation in traumatized patients which they have difficulty describing. High levels of distress tolerated surprisingly well.

3. Frequently #2 progresses to de-repression of traumatic material which pt may or may not have known about. This progression is reproducible within an individual, and is stereotyped across individuals, occurring 10-12 minutes after attainment of med-high coherence.

4. HRV alone appears to frequently activate the traumatic associational network (without necessarily requiring “kindling” with focused questioning about image, emotion, body etc.) which can then be processed with eye movements.

5. There appears to be a “press to express” of affective material.

6. Anti-dissociative effect. Treatment refractory individuals with varying degrees of dissociation are able to contact previously unavailable affect and experience when “pre-treated” with HRV. (This is probably due to #1,2,3 and 4 of “This state is characterized . . . ” above. Typically expressed as “I feel more focused”.

7. Personality Disorders with freeze responses indicative of the most primitive level of autonomic defense (dorsal vagal-presumably secondary to poor tuning of the myelinated Smart vagus c/w insecure attachment) can be engaged with HRV placing them in the autonomic state which supports “peaceful proximity”.

8. Shift in state from adrenergically driven narrow focus of high beta to alpha secondary to HRV results often results in spontaneous resolution of dilemmas i.e. shift in state facilitates information processing.

9. With processing of trauma, pts who initially had the mixed response described in #2 will convert to #1 experiencing with HRV a state of relaxation which is deeper than they have had “in years”.

The following systems and devices are used to detect and induce a therapeutic zone.

### Systems and Devices

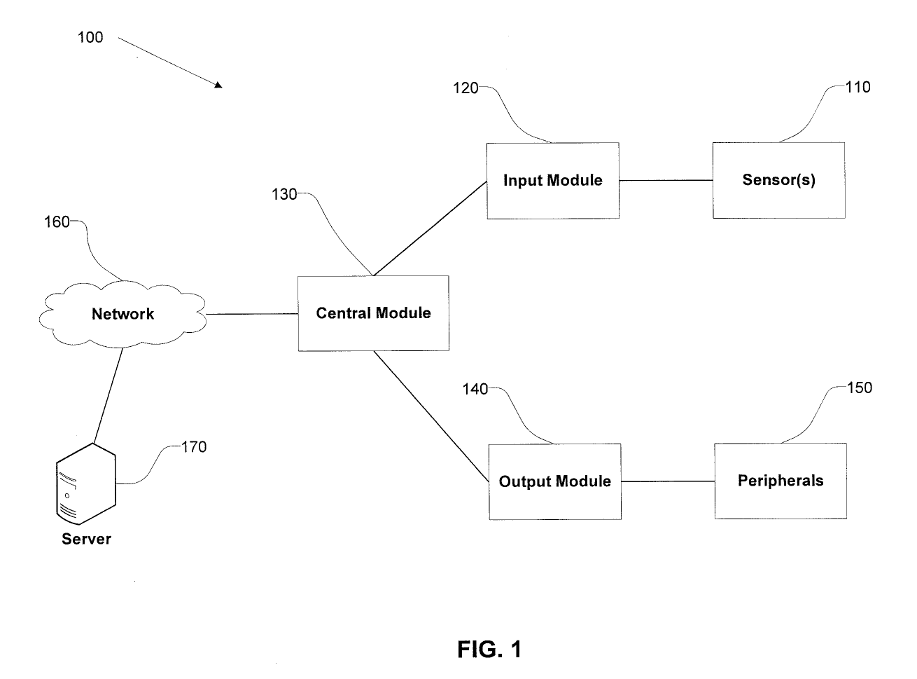

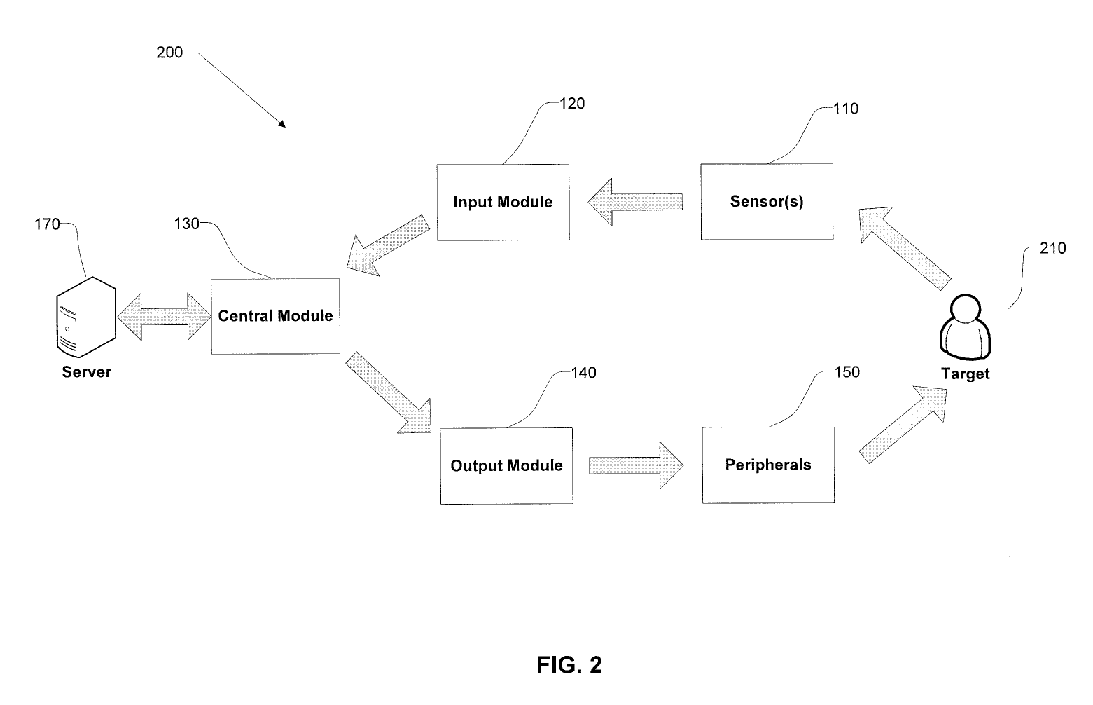

Referring to FIG. 1 and FIG. 2, a System 100 can include at least one Sensor 110, an Input Module 120, a Central Module 130, and an Output Module 140. The Sensor 110 can be configured to detect physiological information of a Target 210 (e.g., a patient). The Sensor can include a photoplethysmograph to sense pulse mounted on, for example, an ear or finger. The Sensor can include an electrocardiogram (EKG) monitor, a respiration belt, skin conductance, and electroencephalography (EEG) monitor. The Sensor 110 can be coupled to the Input Module 120. Via the coupling, the Input Module 120 can receive and process the physiological information detected by the Sensor 110. The Input Module 120 is further coupled to the Central Module 130. Via the coupling, the Central Module 130 can receive and further process the physiological information. The Central Module 130 can be further coupled to the Output Module 140. Via the coupling, the Output Module 140 can receive instruction from the Central Module 130 and generate output accordingly to regulate physiological state of the Target 210. Preferably, the Output Module 140 can be further coupled to one or more Peripherals 150. The Peripherals 150 can facilitate regulating physiological state of the Target 210.

The Central Module 130 can be preferably connected to a Server 170 via a Network 160. The Network 160 can include, but not be limited to, a wired network, a wireless network, a local network, an external network, or the Internet. The Server 170 can provide more information (e.g., statistics) to the Central Module 140 to assist analyzing physiological information and/or regulating physiological state of the Target 210; the Server 170 can also provide storage and/or backup service to the Central Module 140.

As illustrated in an Arrangement 200 in FIG. 2, physiological information is first detected and retrieved from the Target 210. Physiological information is then passed and processed by the Central Module 130 via the Input Module 120. The Central Module 130 can establish two-way communication with the Server 170 during its processing. When the processing is done, the Central Module 130 can generate and send instructions to the Output Module 140, which then generates output signals accordingly. The output signal can be applied onto the Target 210 to regulate the physiological state of the Target 210. Optionally, the application of output signal onto the Target 210 can be done via one or more Peripherals 150. Thus, in the Arrangement 200, the System 100 can be configured to interact with the Target 210 in a bilateral fashion. U.S. Pat. No. 8,066,637 by Childre et al. entitled “Method and Apparatus for Facilitating Physiological Coherence and Autonomic Balance” describes method and apparatus for determining the state of entrainment between biological systems which exhibit oscillatory behavior such as heart rhythms, respiration, blood pressure waves and low frequency brain waves based on a determination of heart rate variability. Devices for monitoring heart rate variability can be available from Thought Technology (Westchester, N.Y.), and Helicor, Inc. (New York, N.Y.) (e.g. Stress Eraser).

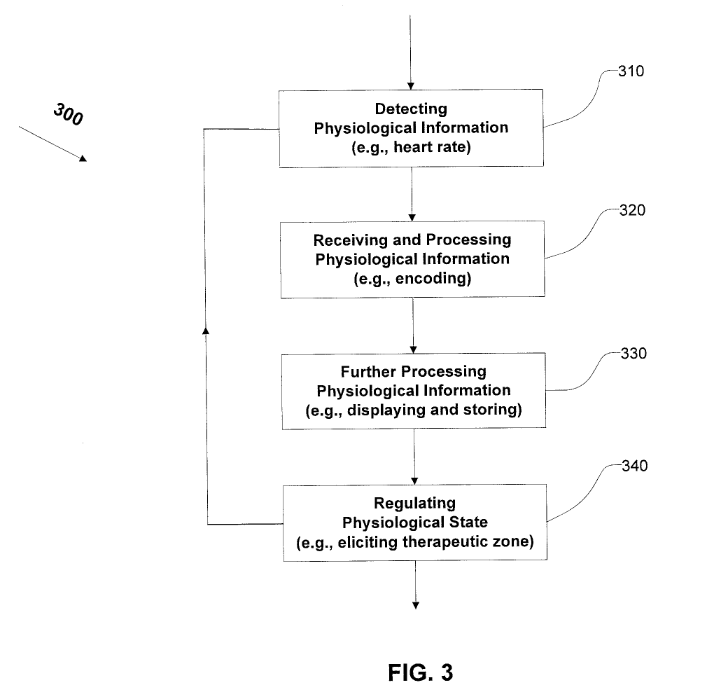

An exemplary Process 300 of the Arrangement 200 is illustrated in FIG. 3. At Step 310, physiological information (e.g., heart rate) is detected by one or more Sensor 110 from the Target 210. At Step 320, the physiological information is received and processed (e.g., encoded) by the Input Module 120. At Step 330, the physiological information is further processed (e.g., displayed or stored) by the Central Module 130. At Step 340, output signal is generated by the Output Module 140 to regulate physiological state (e.g., elicit therapeutic zone) of the Target 210. The steps in the Process 300 can be configured to repeat one or more times as needed to achieve the desired result.

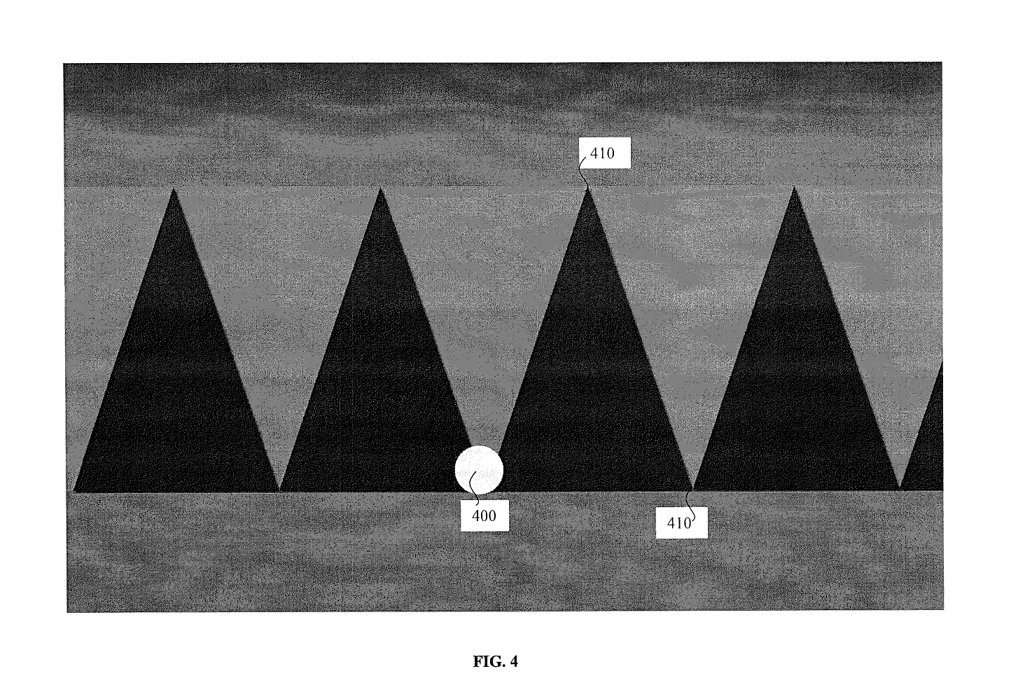

For example, a target can sit in a chair and view a monitor screen providing instructions relating to breathing rate. FIG. 4 is an example display. The breathing pacer object 400 moves to position 410 then position 420 and will continue to move over the displayed triangles. The target attempts to synchronize their respiration with the object so that, they inhale while the object is rising and exhale while the object is falling (i.e. inhale as the breathing pacer object moves to 410, and exhale as the breathing pacer object moves to 420).

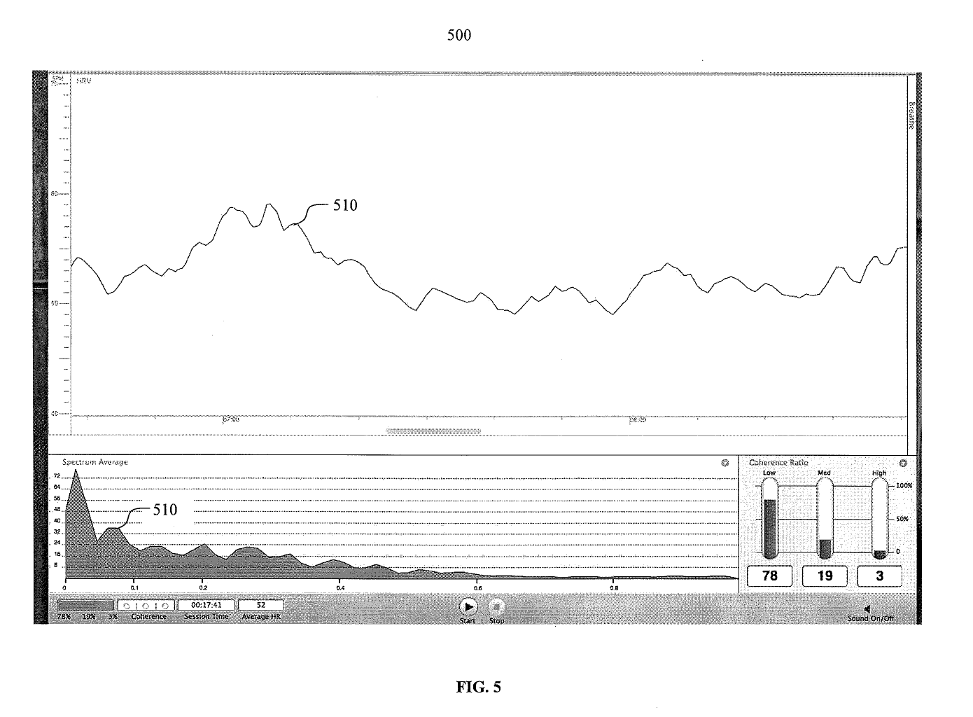

The central module can simultaneously monitor the output of the input module (e.g. the target's heart rate variability). Alternatively, a clinician using the system can monitor on a separate display. FIG. 5 is a plot 500 showing the time domain series 510 of the magnitude of the heart rate variability and its rhythm. 520 is a frequency domain representation of the same data (e.g. the time domain series is has been processed by a Fourier Transform to yield a power spectrum). The central module can adjust the movement of breathing pacer object 400 to identify the breathing rate that yields a large amplitude variation in heart rate variability. This rate represents the resonant frequency of the cardiovascular system.

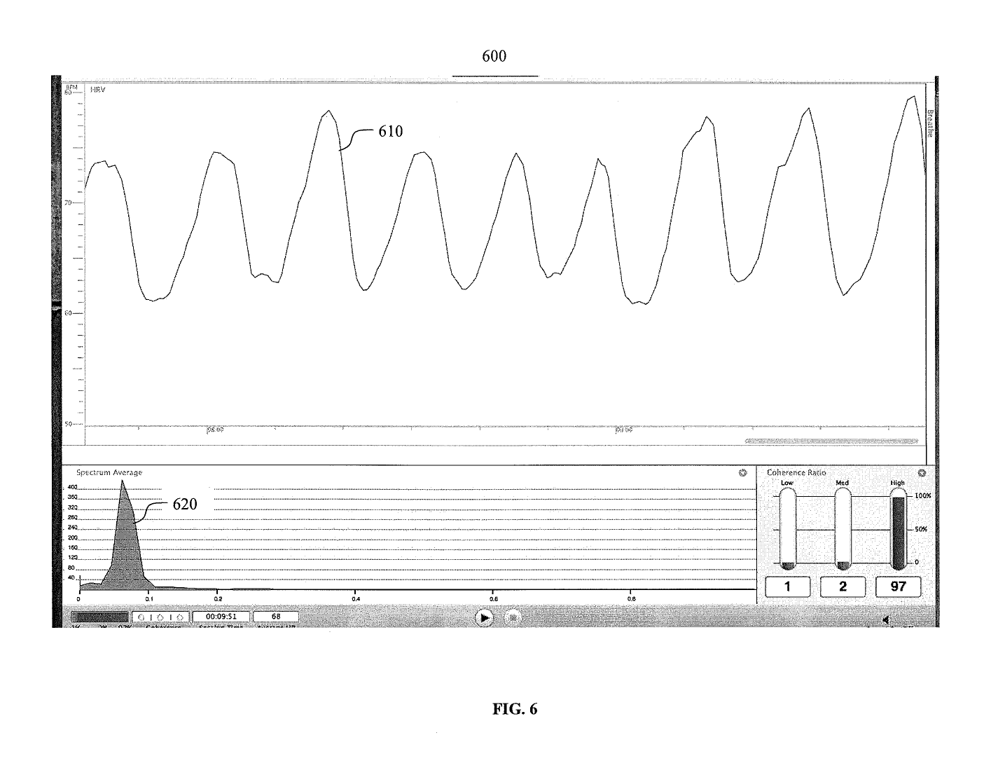

The central module can adjust the movement of breathing pacer object 400 to a resonant frequency which can be, for example, between 4 and 7 breaths per minute). The target can synchronize their breathing with the pacer object. FIG. 6 is a plot 600 illustrating a time domain series 610 of the magnitude of the heart rate variability and rhythm of a target after the target has been breathing at resonant frequency for several minutes. The frequency domain series 620 will show a single high amplitude peak.

Once the target has reached the resonant frequency the target continues to breathe at the paced rate until they reach a therapeutic zone. This may take 8-12 minutes. Targets with unresolved psychological trauma will exhibit an abrupt loss of the sinusoidal wave pattern despite continued respiration synchronized with the pacer. Physical signs such as muscle tension, posture facial expression, motor activity sweating, pupil dilation, etc. may also be used to determine if the target has reached a therapeutic zone. Psychological status can be assessed at four minute intervals (e.g. though content, somatic sensations, anxiety, etc. Having entered the therapeutic zone, physical and psychological assessment can demonstrate the spontaneous emergence of previous traumatic experience.

For individuals who manifest emergence of previous traumatic experience the procedure continues with the addition of bilateral stimulation. (Prior to beginning bilateral stimulation the clinician may opt to ask questions about the experience of the trauma e.g. images, sensory experience, somatic sensations, cognitions, affect, in order to ensure full activation of the neural network storing the experience.)

The breathing pacer being shown to the target is replaced by an object with back and forth motion across the screen at a variable rate determined by the output module or clinician (e.g. Go With That Bilateral Stimulating Software, Neurolateral etc.) The rate can be between 40 to 60 passes per minute for 20-40 passes. The target can be instructed to track the object visually. Horizontal saccadic eye movements can be induced. (Alternatively the eye movements may be induced by the clinician using simple hand movements.)

Because attentional processes are tightly linked to oculomotor movements, sharing a common functional neural network originating in the paritetal cortex, the induction of saccadic eye movements repeatedly re-directs attention, thus disrupting the over-consolidated traumatic memory. (Traumatic memories characteristically manifest “over-consolidation,” meaning that they are recalled from a single psychological viewpoint, in contrast to a non-traumatic memory. This aspect makes them very resistant to integration into semantic memory networks.)

Following a set of eye movements, the target can arrive at a new “attentional set” experiencing the trauma from a different perspective that usually includes strong affect and the associated somatic sensations. After being given time to briefly describe the new perspective, another set of horizontal saccadic eye movements are induced. Saccadic eye movements are quick, simultaneous movements of both eyes in the same direction. This results in arrival at a new attentional set (and precludes prolonged exposure to the affect of the previous attentional set that might induce fear thus reconsolidating and reinforcing the traumatic memory).

As the procedure continues the individual will arrive at attentional sets that are “benign” in that they are not associated with strong affect or somatic sensations. (Eye movements have been shown to decrease the vividness and emotionality of memories.) The individual will also manifest integration of elements of the traumatic experience into existing memory networks.

The clinician then redirects attention to the original trauma to identify aspects that continue to evoke strong affect and somatic sensations. Sets of eye movements are repeated until there is an absence of somatic sensation associated with the event.

This procedure appears to simulate the memory processing that occurs during Rapid Eye Movement (REM) sleep. REM sleep is characterized by a decrease in nor-epinephrine transmission and widespread cortical activation and coherence. The net result is the abstraction and assimilation into semantic memory networks of experience, with a loss of the emotional charge. Because of persistently high levels of sympathetic/nor-epinephrine activity in states of traumatic stress, it appears that REM processing is blocked.

### EXAMPLE 1

### Physiological Psychotherapy—Opening the “Trauma Window” in High Achievers

This example describes the use of heart rate variability biofeedback (HRV) in the practice of psychotherapy, including the emergence of psychological trauma. The techniques described are used to optimize the efficiency and effectiveness of the therapy hour. The clinical case of DG, a successful business executive who was able to significantly improve his level of function, is used to illustrate critical points of the system. “I never thought I'd be comfortable enough to tolerate the discomfort of getting to the root cause of my problems.” (D.G., Senior Business Intelligence Analyst).

As was described above, a subject not being in the “right mood” or “frame of mind” or being anxious, fearful, or overwhelmed hinders the ability to engage and benefit from treatment. For example a person may be so anxious and preoccupied that she can't focus effectively. The clinician is then abruptly confronted with the biological reality that physiological state determines the range of possible functions. If you're falling asleep you can't learn, and if you're body is in a state of fight/flight or freeze, you can't process feelings.

HRV is used to regulate physiological state in the “real time” of the psychotherapy hour in a variety of ways. Clients are instructed to use HRV immediately before a session while sitting in the parking lot, or in the waiting room. By beginning a session with an optimal level of arousal and focus, the therapy hour becomes much more efficient and productive.

Using HRV to regulate physiological state during a session is often an experience that neither therapist nor client will forget. Particularly in “affect oriented” psychotherapies, it is not unusual for levels of arousal and affect to rise precipitously. Sometimes it is impossible for the therapist-client dyad to modulate the arousal, and an overt defensive physiological state of fight/flight or freeze is triggered. (Of note is that with “freezing”/dissociation the client may simply “fade away” in a manner that is very subtle, but very detrimental.) At this point the psychotherapy process has stopped, and it is critical that the therapist recognize the interruption, and act decisively.

Once a defensive physiological state has been precipitated, the clinical focus needs to shift, both for therapist and client. The mental content that triggered the defense becomes secondary. Re-establishing a balanced non-defensive state characterized by feelings of safety is paramount. The therapist should clearly articulate the proposed intervention and its purpose, e.g., “Why don't we do some breathing (HRV) to help you feel more safe?” Because of the nature of the defensive state, this suggestion is often met with resistance which is quite vigorous, e.g., “screw breathing!” Proceeding with gentle but firm insistence is necessary, and will usually be rewarded.

One technique for “insisting” is for the therapist to begin paced breathing in synch with the client. This helps to overcome resistance through behavioral modeling that speaks directly to the client's right hemisphere, bypassing the left hemisphere, which is now “beyond words.” It also emphasizes therapist commitment to the intervention, and decreases feelings of self-consciousness while promoting feelings of connection to a “regulated other.”

If deep breathing is a flashlight, then breathing at the resonance frequency with HRV is a laser. With resonance (equals coherence) comes power. Using HRV a client can shift from a state of terror to one of relative calm in fifteen minutes. For a therapist who is used to “talking people down” it is empowering to have such a potent physiological clinical tool. For the client, particularly if he or she sustained developmental trauma, it may be the first experience of rapid, dramatic relief from extreme distress. In a state of terror, safety feels impossibly far away. To learn that a feeling of safety may be rapidly reached through deliberate action by oneself is extremely empowering. (That you were helped to get there by a caring “attentive other,” thus helping to remediate Attachment deficits is an added benefit.)

As affect and arousal continue to ebb and flow following psychotherapy, HRV is used in between sessions to self-regulate. This helps the client maintain a state that facilitates continued processing of the therapeutic material. In this way HRV is a true “force multiplier.”

Unfortunately the road to greater stability with HRV may be littered with obstacles. As clients move into a state of balance and greater resilience, they will often be immediately challenged to process their “unfinished business.” The clinical scenario is remarkably stereotyped and typically unfolds as follows.

### Heart Rate Variability and the “Trauma Window™”

The client begins using HRV and within a few minutes is able to attain medium to high coherence. Coherence here refers to a state of physiological balance, marked by relaxed even diaphragmatic breathing, and an optimal oscillation in heart rate, in synchrony with breathing, and enhancing autonomic balance.

As parasympathetic tone increases bowel sounds often become audible. The client begins to appear more relaxed—dropping shoulders, sitting back in the chair, relaxing facial muscles, etc. When asked about thoughts and bodily sensation at 5 minutes, she will report slowing of thoughts and relaxation in the body. So far so good.

There is a window from 8 to 12 minutes after attaining medium to high coherence during which there is a significant shift in physiological and psychological state in certain individuals (the “Trauma Window”). Previously attained heart rate coherence will degenerate into a more irregular pattern. The steady progression to greater relaxation is interrupted. The client begins to enter a “mixed state” of relaxation and distress, with a spectrum of clinical signs and symptoms.

On the subtle end of the spectrum clients may report being “bored,” or having vague somatic symptoms, e.g., tingling, muscle twitching or abdominal sensations. Often there is an urge to “do something else.” If encouraged to continue with the HRV, some clients will pass through this state and return to a state of heart rate coherence and relaxation. If not encouraged, most people will discontinue HRV. “I don't know, I didn't really like it (the HRV)”.

For some clients who continue the HRV session, the “mixed state” will intensify with increasing signs of restlessness, agitation, and worry. When asked, clients will report a mixture of apparently incompatible feelings and sensations, e.g., “I feel relaxed but wound up.” “I'm sleepy, but there's all this energy in my body.” Inability of clients to label their feelings and sensations in a coherent, satisfying way is typical.

As the “mixed state” continues, physical pain may emerge and become localized. Previously vague symptoms become more intense and clearly defined, e.g., “tightness in my throat.” At the dramatic end of the spectrum the client will have a flashback, or re-experience a traumatic event with a complete complement of images, thoughts, affect and sensation. Although the flashbacks that occur during HRV are challenging, they are generally tolerated better than would be expected based on clinical experience. Presumably this is a reflection of the more resilient state associated with increased HRV.

While it may seem incongruent to be discussing trauma in the context of high achievers, experience teaches that it is a mistake to equate achievement with psychological well-being. Across the spectrum, from performers to athletes to executives, the drive to excel is often a response to traumatic experience. In turn, unresolved traumatic experience ultimately limits achievement.

If something that is supposed to help me to feel better makes me feel worse, then why should I do it? This is an excellent question, which has a satisfying answer for most of the interventions therapists propose, e.g., quitting smoking or starting an exercise program. Unfortunately for most people who try HRV for “stress management” and encounter the “Trauma Window,” this question has not been adequately answered. In fact for people on the subtle end of the spectrum their encounter with the “Trauma Window” never rises to the level of consciousness. They experience mild discomfort and discontinue the session concluding, “it's not for me.” It is very common for people to develop an aversion to HRV itself as a result of encountering the “Trauma Window” without conscious awareness of having done so. Addressing this phenomenon is critical to realizing the potential of HRV in clinical practice. How can we understand the “Trauma Window” in a way that makes sense, and will provide therapists and clients with the fortitude to tolerate the bumps on the road to increased stability and balance?

The term “Trauma Window” is a convenient clinical shorthand which refers to the temporal window of 8 to 12 minutes during HRV described above. It also refers to a “window into the trauma” of a person's life that results from being in a particular physiological state. Such “trauma” spans a broad range from the horrors of combat to humiliation in the schoolyard. In general the “Trauma Window” can be considered a physiological state that produces “release phenomena” familiar to practitioners of a variety of disciplines including Biofeedback, Neurofeedback and Body-work. One factor that seems to distinguish the phenomenon in HRV is the high frequency and regularity with which it occurs. This difference can be understood with appeal to the underlying physiology.

Example 3 below includes prescribing guidelines, showing how one begins the breath and HRV training, and how one can tailor the client's HRV practice outside the session depending on how much trauma and distress actually emerges in the “Trauma Window™.”

Breathing at the resonance frequency of the cardiovascular system (HRV) leads to a state of moderate arousal, with balanced activation of the autonomic nervous system, and increased alpha waves. These are the essential attributes of the “arousal portal” described by Les Fehmi, in which attention is fluid, and multiple attentional states co-exist (Fehmi, 2010). Diffuse Focus attention is characterized by a broad awareness of the self and world, without particular focus on any one thing. Narrow Focus attention is more like a spotlight that focuses on one thing and ignores everything else outside of its beam. In the “arousal portal,” both of these states can exist simultaneously giving rise to a very powerful way of attending to experience.

Narrow Focus attention has been characterized as an emergency mode that facilitates survival. This mode is associated with increased sympathetic arousal and high frequency (beta) brain waves. The soldier who “Narrow Focuses” on getting to safety, rather than on his feelings about being wounded, is much more likely to survive. And under less dramatic circumstances, anyone may learn that by “Narrow Focusing” painful feelings can be ignored.

For example during a painful divorce a man may “Narrow Focus” on his work to avoid feeling overwhelmed. The resulting relief reinforces the use of Narrow Focus which may then be used to deal with other difficult feelings. If his use of Narrow Focus becomes a habit, however, there will be an accumulation of “unfinished business.” There will also be the wear and tear of a chronically over-activated sympathetic nervous system. Seeking relief, he finds his way to HRV and begins training

As he shifts to the more balanced state, his sympathetic arousal decreases. His brain waves shift from high frequency localized processing to lower frequency, more global and synchronous processing. And simultaneously his attention begins to shift to Diffuse Focus. He begins to feel more relaxed and present. So far so good.

The attentive reader can anticipate that as the man enters the “Trauma Window” this individual's discomfort will rise, and the reader will not be surprised when he tearfully says, “I'm scared, I don't want to grow old alone.” Based on the functional model, emergence of powerful feelings is predictable and expected. In fact it presents a tremendous opportunity for healing. Having set the stage, the prepared clinician can utilize her therapeutic technique of choice to facilitate continued processing. Clinical observations and experience described herein indicates EMDR/bilateral stimulation (e.g., HRV biofeedback using the systems and methods described) is particularly effective, in resolving such traumatic experiencing.

### A Case Study: DG

DG is a 45 year old divorced Senior Business Intelligence Analyst with bipolar disorder, who presented for treatment to “keep me out of trouble.” Having sustained multiple episodes of mania and depression over 25 years, he had resigned himself to his “biological destiny.” Pessimistic about improving his performance, he was more focused on not losing function. Given his demanding role as liaison between Senior Management and the Information Technology (IT) Department, this concern was easy to understand. Like many high achievers he used his considerable strengths to “work around” his deficits which were therefore not readily apparent. For example by virtue of working in IT, it wasn't noticed that he communicated almost entirely via computer, thus obscuring his near phobic avoidance of phone calls. Similarly his excessively long work hours insulated him from criticism for not attending social work functions. Ultimately his adaptations left him exhausted and with a sense of shame regarding his never ending avoidance.

Psychotherapy was characterized by an intellectual style that kept discussion of emotional issues on a cognitive level. Despite content suggesting considerable distress and functional compromise, the associated feelings were largely missing. He seemed to be fearful of affect in general, which he tended to avoid. HRV was initiated with the goal of improving his capacity for self-regulation. Perhaps if he was more confident in his capacity to self-regulate, he would be better able to tolerate the physiological changes associated with feelings. And with increased capacity to experience feelings there would be less need to avoid uncomfortable things in his life, past and present.

During his initial training with HRV, DG was able to attain 50% combined medium and high coherence. After approximately 8 minutes he developed “body tingling,” an increase in his baseline tinnitus, and “tense thoughts.” With encouragement he continued and at 15 minutes he described his thoughts as “quiet.” The tingling decreased and the tinnitus returned to the baseline level. After finishing the training he reported that he had felt very uncomfortable and “like I was looking over the edge.” He was perplexed as to why he was so uncomfortable when he was “basically feeling calm.”

The following observations about his response to training were discussed before he left the session. He appeared to have difficulty relaxing deeply (which he acknowledged) and he seemed to have “unfinished business” in the form of feelings which he tried to keep at bay. In addition it was likely that HRV could help him tolerate and release those feelings, with increased mood stability as a likely result. The client then left and went to a local supermarket where he burst into tears. (Having been briefed about the possibility of further “release,” he “rode it out” and reported these events at this next session).

He reported that in 25 years of treatment he had never had that type of reaction. Waves of grief washed over him leaving him with a profound sense of relief. With the self-directed tool of HRV to help him manage his feelings, he had hope for the first time in years. He realized that he had been living with unacknowledged terror of his illness since his first episode. He feared that any strong feeling meant he was going to have a manic or depressive episode, and that all he had achieved in life would be lost. As a result he avoided strong feelings and his personal and professional function was severely compromised. Having experienced waves of grief that were nothing like clinical depression, he felt that he didn't have to fear his feelings as he had in the past.

He began using HRV exercises outside the office, and with a reduction in fear of feelings, experienced a rapid acceleration of progress. Example 4 provides instructions for the client for HRV practice outside the session.) For example he was able to contact his feelings of fear, and of being a burden, engendered by his alcoholic father's irritable response whenever he asked for help. As an adult this residual fear made the lack of visual feedback with phone calls intolerable. To his astonishment, after having released these feelings, he began making phone calls spontaneously. “You have to realize, I don't do this. I don't call anybody ever. I can count on one hand the number of people I will call on the phone. I can't believe I'm doing this. It didn't seem scary at all. I wanted to talk to her so I called her. Great, but damn weird. These are positive things but they are not things I am used to.”

The nature of the psychotherapy work changed to helping him to reorient and adjust to his new identity including new feelings and behavior. “I'm not used to feeling good if it's not an aberration” shifted to “I'm no longer waiting for the other shoe to drop.” Ongoing psychotherapy helped him to differentiate between ordinary feelings and symptoms of a major affective episode e.g. sleep quality. In addition reinforcement for feeling a spectrum of feelings led to a significant reduction in shame. “I don't feel like I'm weird anymore.”

The ability to make phone calls was the leading edge of a generalized increase in his capacity to express himself and engage with others. No longer locked into Narrow Focus mediated avoidance of feelings, he decreased his work hours by a third, and began to see new social and professional opportunities. Ultimately this led to moving to another city to pursue a romantic relationship, confident in his ability to secure a challenging professional position. “I can be happy, and I owe it to myself to try.” “I'm having fun in my life and I like it.” “I'm not going to collapse if bad things happen.” “Life is easier”.

### HRV as a Clinical Tool

HRV is a powerful clinical tool that facilitates access to a physiological state characterized by flexible attention and resilience. In this state, psychological trauma is available, and its associated discomfort well tolerated. These attributes make HRV an ideal physiological regulator in the practice of psychotherapy.

### EXAMPLE 2

### Physio-Logical™ Psychotherapy—Staying in the Therapeutic Zone

Erin was different that afternoon. Absent was the protective swagger and bravado that allowed her to engage in therapy without getting too close. Cowering in the corner of the overstuffed couch she looked like a frightened little girl, not the street savvy twenty-something that she was. Vulnerable. Frozen. Stunned?

Erin was fighting the good fight. Born to a crack-addicted mother, she lived in a series of foster homes before moving to suburbia to live with her grandmother as a world-weary twenty year old. Her father's incarceration for manslaughter was both a source of shame, as well as a graphic testament to his profound emotional dysregulation. But she was building a life. Community college. Steady boyfriend. And she had a dream.

The chaos of her early life experience was imprinted in the function of her nervous system. Every day was a battle as she tried to tame this inner chaos. Some days she succumbed, and like her mother, sought refuge in the numbing of intoxication. Some days she hurt those close to her, and with shame would pick up the pieces the next day. But she understood that she was engaged in an epic battle, and took pride in her victories. Something was different today.

Psychotherapy can be extremely powerful, however the progress is often uneven. Frequently clients are not in the “right frame of mind” to engage in the challenging work of self-transformation. For example a man may be so preoccupied by work that he can't focus effectively. Or in talking about emotionally charged issues a woman may become overwhelmed, frozen and shut down. For psychotherapy to be consistently effective it is imperative to recognize that being in the “right frame of mind” is a function of the body's physiological state. The therapist who learns to identify, monitor, and co-regulate the client's physiological states will enjoy consistently better outcomes. Why?

The essence of emotion is physical. Emotions are “of the body”. Long before mammals evolved linguistic competence emotions were facilitating survival. The “surprise” triggered in the gazelle by the sound of a lion at the watering hole precipitates a cascade of coordinated physiological changes. The head is raised, and turned toward the sound. Heart rate momentarily decreases to facilitate sensory processing, and the pupils constrict to see the threat more clearly. When the gazelle sees that a lion makes the sound, this “orienting response” gives way to the “fight or flight response” and a new cascade of physiological changes occurs. Heart rate, blood pressure and respiratory rate increase. Blood flow is redirected from the gastrointestinal tract to the skeletal muscles. Muscles increase in tension, preparing to “fight or flee”. With these changes in physiology come important changes in function.

Physiological state determines the range of functions that are available to a person at that moment in time. Some functions will be facilitated, and others inhibited. For example the physiological changes associated with the emotion of “surprise” that are called the “orienting response” set the stage for detecting a threat, or being delighted by the approach of an old friend. Going to sleep is not an option. Alternatively the emotion of “fear” with the accompanying physiology of the “fight or flight” response sets the stage for fighting off an attacker, or running to catch a bus. However digesting a meal, or getting an erection, are not options. Why is this important for psychotherapy?

Psychotherapy is a social function that requires cooperation, sustained engagement, flexible attention and contingent communication. As such, certain physiological states are incompatible with psychotherapy e.g. intoxication or psychosis. If a woman is intoxicated she is unlikely to be able to sustain the required attention or engage in true contingent communication. Thus client and therapist will be talking at each other, rather than with each other. Alternatively if a man has a paranoid psychosis and believes that the therapist is trying to harm him, the possibilities for change are very limited. In these situations therapy stops, and therapist intervention is appropriately aimed towards restoration of a more functional physiological state e.g. a cab ride home and sleep, or psychiatric evaluation. Intoxication, psychosis, and severe depression are all examples of physiological states that are incompatible with psychotherapy and not readily reversed in the therapy hour. Are there any states that can be reversed in the “real time” of the psychotherapy hour?

Erin wasn't moving very much. She didn't make eye contact and her gaze was fixed. Her breathing was shallow and she didn't appear to be engaged with her environment. She appeared frozen, stunned. The therapist wonders, “What happened, what's her story?” In contrast, the physiologically attuned therapist wonders, “ what state is she in, and how do I help her move into a state where she can communicate and process what happened?”

From an evolutionary perspective immobilization was the first defense to develop in animals. An iguana whose brain perceives threat freezes his body to avoid detection by the hawk. This action is mediated by the Autonomic Nervous System (ANS) which has two major components, the Sympathetic and Parasympathetic divisions. The ANS acts to couple the brain and the body, thus ensuring a coordinated response. According to Stephen Porges' Polyvagal Theory, the specific ANS mechanism responsible for immobilization behaviors is called the “Vegetative Vagus” nerve, part of the Parasympathetic Nervous System. (“Vegetative” means involuntary in science speak-the iguana doesn't have to think about freezing, it's an automatic reflex. “Vagus” in Latin means “wandering” which is what the nerve appears to do as it courses through the chest and abdomen. It's a huge nerve, second only in size to the spinal cord.)

The enduring presence of the immobilization response in humans is captured by such colloquial expressions as “scared stiff” or “frozen in fear”. We see it when an inexperienced public speaker steps up to the podium and is unable to talk. Or more dramatically in a bank robbery when a person remains standing despite repeated commands to “get down on the floor”. And in psychotherapy we see it in the surprisingly common phenomenon of dissociation, which is frequently subtle enough to not be identified by either therapist or client. Failure to identify even subtle dissociation leaves the client in a state of compromised internal engagement with emotions and memory, and external engagement with the therapist. Therapy effectively stops, even if the client continues to maintain superficial engagement.

Using the familiar therapeutic interventions of a comfortable quiet room, simple statements and questions delivered in a soft empathic tone, with a slow rhythm, and intermittent non-threatening eye contact, Erin was coaxed out of her frozen state. With hesitation she shared her story.

Ever since she was a little girl living in a foster home, Erin had dreamed of being a mother. Of being the kind of mother that she longed for but never had. Each lapse of attunement, or petty cruelty, or indifference in her care shaped her vision of how she wanted to be different as a parent. This dream helped to sustain her as she navigated the inner chaos of her “epic battle”. Thus when she learned that she might be infertile she was devastated. That she had contributed through sexual promiscuity and repeated pelvic infections only fueled her self-loathing and despair. As she recounted her story, she was very briefly in the “therapeutic zone”, able to experience and share her despair, her tears. Then without warning she shifted state again.

“I bet I could take you in a fight.” Taunting, “You're just a skinny white guy.” And several moments later, “I'm leaving”.

The second defensive response to evolve in animals was mobilization. Once again the ANS provides the coupling of brain to body to coordinate response, but this time through the Sympathetic Nervous System (SNS). When activated, the SNS gives rise to the familiar “Fight or Flight” response with increased heart rate, respiratory rate, blood pressure, secretion of cortisol and other changes which optimize chances for survival. Where immobilization might get you out of some jams, taking action through attack or just plain getting away is more likely to get you out of danger. But why was the Fight or Flight response triggered in Erin, and how could she be helped back into the “therapeutic zone”?

To understand Erin's predicament requires the “piece de resistance” of Stephen Porges theory, the Social Engagement System (SES). The most recent defensive mechanism to evolve in humans doesn't necessarily seem to be involved in defense at all. However, what ultimately protects us better than any physical attribute is our ability to engage and cooperate with others. Social engagement allows us to meet our basic needs, including protection, better than we could as individuals. And for the newborn the ability to engage others is absolutely essential for survival. How does the Social Engagement System work?

The most recent development of the ANS is the “Myelinated Vagus” nerve. (To facilitate discussion this will be called the “Social Vagus”.). The Social Vagus is also part of the Parasympathetic Nervous System however it functions very differently from the Vegetative Vagus (immobilization) described earlier. The Social Vagus integrates the function of muscles in the head and neck critical for social behavior e.g. facial expression, vocalization, eye opening and tracking, with calm visceral states conducive to social engagement. The result is a system that regulates physical and psychological distance as appropriate for our needs and the safety of the situation. This concept is easily illustrated by the predicament of the hungry infant.

Lacking necessary nutrients the hungry infant's viscera, including heart, are “agitated”. Her heart rate rhythm is erratic. Unable to walk to the refrigerator and make a sandwich, the infant activates her Social Vagus. She turns her head, opens her eyes, draws her face into a grimace, and vocalizes her distress in a series of wails of increasing urgency. This is a coordinated and highly effective set of actions that engages all but the most impaired caregiver. The Social Vagus then coordinates the rather complicated sequences of sucking, swallowing and breathing while she feeds. As she begins to settle her face relaxes and her heart begins to enter a calm state characterized by increased and rhythmic variability of her heart rate. (If measured, her heart rate would look like a smooth, regular, undulating wave.) Satisfied and secure in her mother's arms she closes her eyes and turns her head away, thus communicating her need to not be stimulated for a period of time. The Social Vagus engages mom, then disengages with mom psychologically while remaining physically close.

As she continues to accumulate social experience the function of her Social Vagus/SES will become much more nuanced than the reflex described above. The SES will be “tuned” by experience and she will become more adept at the dance of interpersonal relationships. A well-tuned Social Vagus facilitates states of excitement, both positive and negative while maintaining proximity. Adults can vehemently disagree and remain near each other without freezing, physically attacking or fleeing e.g. imagine a good day in the US Senate. In contrast, if an individual's formative experience is characterized by interpersonal chaos and strife, their SES will not support this function of “peaceful proximity”.

After allowing herself to feel sadness in the presence of another, Erin felt extremely vulnerable. Lacking a well tuned SES that would allow her to remain nearby despite discomfort, she was catapulted into a state of fight or flight. Not having a more adaptive mode of functioning she instinctively “defended herself” with the next best tool she had been given by evolution—the Sympathetic Nervous System. (Erin's experience is echoed by a recent study in which emotionally charged video clips were presented to Borderline Personality Disorder (BPD) patients and controls. The BPD patients ended in the fight or flight state, the controls in a state conducive to social engagement.) Unfortunately this removed her from the “therapeutic zone” which requires social engagement. The challenge for the physiologically attuned psychotherapist is bringing Erin back into the “therapeutic zone” as rapidly as possible. How might this be accomplished?

A brain based threat detection system called Neuroception has been postulated. Processing sensory and visceral feedback (gut feelings) the brain assesses the safety of a situation. If it is deemed safe, the Social Vagus will facilitate social engagement through motor output in the head and neck e.g. turning, making eye contact, smiling and speaking It will also put the viscera in a calm state. Specifically this means a state where the variability in the heart rate is high. So if you feel safe, your heart rate variability will be high. A leads to B. Might the reverse be true i.e. that increasing heart rate variability leads to feeling safe?

An important feature of the nervous system is its bi-directional nature. For example activation of the sympathetic nervous system leads to muscle tension (“top-down” influence). And muscle tension maintains activation of the sympathetic nervous system (“bottom-up” influence). So when we want to relax, we stretch our muscles (Yoga), decrease sympathetic output, and smile again. We change our body, to change our brain, which changes our mind.

HRV is a powerful clinical tool that can be used to “turn on” the Social Engagement System. HRV trains an individual to use breathing to stimulate her cardiovascular system at its unique resonance frequency for a sustained period of time. The resonance frequency is that rate of breathing, usually between 4 and 7 breaths per minute, which produces the largest heart rate oscillations, that is the greatest heart rate variability. This property makes it significantly more potent than standard “Deep Breathing” techniques. If deep breathing is a flashlight, breathing at the resonance frequency is a laser—much more powerful, and with new properties. Breathing at the resonance frequency stimulates pressure sensitive vascular baro-receptors, whose output ascends via the vagus nerve to modulate CNS function. Sympathetic nervous system tone is reduced, stress hormone secretion decreased, and the brainwaves shift towards the “relaxed, alert” alpha state.

Psychologically this translates into a client who is “relaxed and ready” with an optimal level of arousal and focus. Internal dialog is reduced, feelings of well-being increased, and receptivity to input enhanced. The individual is more present and available for Right hemisphere to Right hemisphere emotional regulation by attuned others. Social behavior increases spontaneously. By placing the body (heart) in a state that is associated with safety and social engagement, the person is strongly pushed towards the corresponding brain and mind states of safety and social engagement. A change in the body, changes the brain, which changes the mind.

HRV can be used to regulate physiological state in the “real time” of the psychotherapy hour in a variety of ways. Clients can be instructed to use HRV immediately before a session while sitting in the parking lot, or in the waiting room. By beginning a session with an optimal level of arousal and focus, it is possible to “hit the ground running” making for a much more efficient and productive session.

Using HRV, a client is induce to shift from a state of terror to one of relative calm in fifteen minutes. For a therapist who is used to “talking people down” it is empowering to have such a potent physiological clinical tool. For the client, particularly if he or she sustained developmental trauma, it may be the first experience of rapid, dramatic relief from extreme distress. In a state of terror, safety feels impossibly far away. To learn that a feeling of safety may be rapidly reached through deliberate action by oneself is extremely empowering. (That you were helped to get there by a caring “attentive other,” thus helping to remediate Attachment deficits is an added benefit.)

As affect and arousal continue to ebb and flow following psychotherapy, HRV is used in between sessions to self-regulate. This helps the client maintain a state that facilitates continued processing of the therapeutic material. In this way HRV is a true “force multiplier.” So what about Erin?

Erin had abruptly left safe social situations before. Usually this resulted in feelings of emptiness and despair. And when she was reminded of this, she reluctantly agreed to begin breathing using HRV. In ten minutes she shifted from a state of Sympathetic “mobilization” to one of Social Engagement “peaceful proximity”. And as she shifted states, her cognition improved and she was able to think more rationally. Her interpersonal boundaries became less rigid, and she was able to listen. And what she heard, that she was not able to hear before, changed everything. “Your doctor said you might be infertile.” There was hope. The dream was still alive.

Consistently effective, efficient psychotherapy requires attention to the client's physiological states. Heart Rate Variability Feedback is a powerful tool that facilitates self-regulation of the Autonomic Nervous System. HRV helps clients to enter and remain in the optimal state of Social Engagement for Self-transformation—the Therapeutic Zone.

### EXAMPLE 3

### Prescribing Guidelines for HRV Homework

In the initial evaluation, Resonance Frequency is approximated by guiding the client to breathe at rates between four and seven breaths per minute (e.g., 4, 4.5, 5 breaths per minute). The Resonance Frequency is the breathing rate that produces the largest oscillation or “swing” in heart rate. Then clients are provided with approximately three sessions of breath training and HRV biofeedback at their identified Resonance Frequency. The computer screen includes a breath pacer guiding the client to his or her Resonance Frequency. Following training in the office, many clients will benefit from using heart rate variability (HRV) practice at home. Using a feedback device that displays the heart wave is recommended, because it maximizes time spent at the resonant frequency and also reflects negative emotions as irregular waveforms. Visualizing an irregular waveform makes anger and anxiety easier to identify, thus facilitating Mindful Awareness. The StressEraser™ and emWave™ Desktop are suitable for this purpose. Each device enables the client to breathe with his or her heart rate oscillations, optimizing these oscillations. The amount of HRV home training prescribed depends on how much distress the client is showing in sessions, during the Trauma Window™.

Empty “Trauma Window™”—Clients who pass through 15 to 20 minutes of HRV in the office without emergence of any symptoms suggestive of trauma are instructed to begin using HRV at home for 15 to 20 minutes once a day.

Mild Symptoms in “Trauma Window™”—Clients who have mild symptoms in the office are instructed to use HRV once a day for 20 minutes unless it becomes “too uncomfortable.” Rarely does anyone have trouble complying with this directive.

Moderate to Severe Symptoms in “Trauma Window™”—Clients who experience moderate symptoms in the office are instructed to use HRV for 4 to 6 minutes once a day. This limited use facilitates some relaxation without precipitating trauma. It helps to create a positive association with HRV, sets the stage for entering the Trauma Window in the office, and is “a taste” of what unrestricted use has to offer after trauma has been metabolized.

General Instructions—When using HRV during therapy, some sessions will feel better than others. It is important to “let whatever happens happen” and not try to force a particular experience. A difficult session, like a difficult conversation, doesn't mean that it wasn't helpful. The more one uses HRV, the more consistently sessions (and life) feel “good.”

### EXAMPLE 4

### Device-Based Home HRV Practice—Instructions for Getting Started

emWave™ or StressEraser® are devices that can facilitate HRV practice. emWave™ is a device with colorful LED displays, audio feedback and breathing pacer, and the StressEraser® is portable biofeedback device that uses a finger sensor to convert a pulse reading into an HRV wave. The following instruction are provided to the subject.

Find a quiet, comfortable place to sit uninterrupted for 20 minutes once a day.

Follow the instructions on your emWave™/StressErase®and begin to create the largest smooth wave possible.

Using the emWave™, try to enter and stay in the Green zone (High Coherence). Using the StressEraser®, try to earn three boxes under each heart wave.

If 20 minutes seems like a long time, start with 5 minutes and gradually extend the time.

Maximum benefit tends to occur when used from noon to early evening.

Using immediately before sleep may be too stimulating and disrupt sleep.

Every time feels different. Don't force it. Let whatever happens happen.

Don't sweat the numbers. Regardless of your “score,” benefit results from practice.

Discomfort may be experienced. That's okay, keep going.

It's like a difficult conversation that doesn't feel good, but is helpful anyway.

If it's really uncomfortable, stop and discuss your experience with a therapist.

Using before the therapy appointment, in the parking lot or the waiting room, may be helpful for the challenging work of therapy. Discuss this option with the therapist.

Using between appointments can help to keep the subject balanced and accelerate your progress.

### Benefits and Advantages of HRV Feedback in Psychotherapy

1. The goal of biofeedback, including HRV feedback, is to increase the capacity for self-regulation, i.e., to moderate extremes of physiology and affect. To “turn the heat down”. Therefore if use of HRV feedback results in physiological arousal, and more intense affect, it is doing the opposite of what it is designed to do. At best this response would be considered an “undesirable side effect”, at worst a failure. From the perspective of traditional biofeedback the idea that this response might be used therapeutically is counter-intuitive and non-obvious.

2. The goal of contemporary psychodynamic psychotherapies is to facilitate the experience of intense affect and its concomitant physiological arousal during the therapy hour. To “turn the heat up”. The idea of using biofeedback technology that is designed to moderate extremes of physiology and affect to achieve this result is counter-intuitive and non-obvious.

3. As described in the work of Stephen Porges on the Social Engagement System, when the individual feels safe in a social situation, and therefore comfortable enough to express herself, the result in the body will be a state characterized by high heart rate variability. (“Top-down causality”.) The idea that using HRV feedback to put the body in a state of high heart rate variability would result in a feeling of safety and increased expressiveness (“Bottom-up causality”) (for the purpose of psychotherapy) is non-obvious.

4. The idea that using HRV feedback to increase heart rate variability would result in a state of increased attentional flexibility that facilitates psychotherapeutic processing is non-obvious.

Various implementations of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computer having a display device (e.g., a CRT (cathode ray tube) or LCD (liquid crystal display) monitor) for displaying information to the user and a keyboard and a pointing device (e.g., a mouse or a trackball) by which the user may provide input to the computer. Other kinds of devices may be used to provide for interaction with a user as well; for example, feedback provided to the user may be any form of sensory feedback (e.g., visual feedback, auditory feedback, or tactile feedback); and input from the user may be received in any form, including acoustic, speech, or tactile input.

The subject matter described herein may be implemented in a computing system that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), and the Internet.

The computing system may include clients and servers. A client and server are generally remote from each other and typically interact through a communication network. The relationship of client and server arises by virtue of computer programs running on the respective computers and having a client-server relationship to each other.

Although a few variations have been described in detail above, other modifications are possible. For example, the logic flow depicted in the accompanying figures and described herein do not require the particular order shown, or sequential order, to achieve desirable results. Other embodiments may be within the scope of the following claims. Having thus described at least one aspect of the invention, various alternations, modifications and improvements will readily occur to those skilled in the art. Such alternations, modifications and improvements are intended to be within the scope and spirit of the invention. Accordingly, the foregoing description is by way of example only and is not intended as limiting.

What is claimed is:

1. A system, comprising:
at least one sensor for detecting physiological information of a target;
an input module, coupled to the at least one sensor, for receiving and processing the physiological information of the target;
a central module running on a host computer, coupled to the input module, for further processing the physiological information of the target; and
an output module, coupled to the central module on the host computer, for regulating physiological state of the target.

2. The system of claim 1, wherein the input module encodes at least some of the physiological information of the target received from the at least one sensor.

3. The system of claim 1, wherein the central module displays and stores the processed physiological information of the target.

4. The system of claim 1, wherein the output module is configured to elicit a therapeutic zone from the target.

5. The system of claim 4, wherein the therapeutic zone occurs during a psychotherapy session.

6. The system of claim 1, wherein the physiology information of the target comprises one or more of the following: electromyographic information, electroencephalographic information, electrocardiographic information, respiration waveform, respiration rate, respiration amplitude, blood volume pulse waveform, heart rate, heart rate variability, skin temperature, and skin conductance.

7. The system of claim 1, wherein the output module generates an audio signal.

8. The system of claim 1, wherein the output module generates a visual signal.

9. The system of claim 1, wherein the output module generates a mechanical signal.

10. A method for optimizing a psychotherapy session or shifting a state of social engagement comprising detecting a breathing pattern of a subject and administering to said subject a signal during said session to elicit resonance frequency, wherein a change in said breathing pattern to resonance frequency optimizes said psychotherapy session of shifts said state of social engagement into a pro-social mode.

11. The method of claim 10, wherein said signal comprises an auditory, tactile, or visual stimulus.

12. The method of claim 10, wherein said breathing pattern is changed to a rate of 4-7 breaths per minute.

13. A kit comprising the system of claim 1 and instructions for using heart rate variability to train an individual to use breathing to stimulate the cardiovascular system at its unique resonance frequency for a sustained period of time.

14. A method comprising:
receiving data characterizing a heart rate variability of a target;
determining, from the received data, a psychological state of the target; and
providing, based on the psychological state, feedback to the target to elicit entry into a therapeutic zone.

15. A method comprising:
monitoring a heart rate variability of a target; and
iteratively adjusting breathing pattern instructions for the target based on the heart rate variability thereby eliciting a resonance frequency to shift the psychological state of the target into a therapeutic zone, the resonance frequency being a large heart rate oscillation.
